(19) United States
(12) Patent Application Publication
(10) Pub. No.: US 2026/0157816 A1
(43) Pub. Date: Jun. 11, 2026

(54) SYSTEM FOR POSITIONING MAGNETIC FIELD SOURCES WITH RESPECT TO AN ISOCENTER AND METHOD OF USE THEREOF

(71) Applicant: STARPAX BIOPHARMA INC., Montréal (CA)

(72) Inventors: Dumitru Loghin, Laval (CA); Maxime Latulippe, Montréal (CA)

(21) Appl. No.: 19/413,578

(22) Filed: Dec. 9, 2025

Related U.S. Application Data
(60) Provisional application No. 63/730,232, filed on Dec. 10, 2024.

(51) Int. Cl. A61B 34/00; A61B 34/30; A61B 34/10; A61B 34/20

(52) U.S. Cl. CPC A61B 34/73; A61B 34/30; A61B 2034/102; A61B 2034/2055

(57) ABSTRACT

A method for guiding the positioning of magnetic field sources of a magnetic-field based system, the system comprising a plurality of magnetic field sources oriented towards a support for receiving a subject; it includes receiving positional data for each of the plurality of magnetic field sources; determining a pose of each magnetic field source of the plurality of magnetic field sources from the positional data; generating a computer-rendered model of a target layout of the plurality of magnetic field sources, defining a select pose for each of the plurality of magnetic field sources; comparing the select pose with the determined pose to calculate one or more of a rotation and translation of the magnetic field source; and generating instructions to adjust one or more of a position and orientation of at least one of the plurality of the magnetic field sources from the calculated one or more of a rotation and translation.

The present application claims priority from U.S. provisional patent application No. 63/730,232 filed on December 10, 2024, incorporated herein by reference.

### TECHNICAL FIELD

The present disclosure relates to magnetic field-based medical treatments, and more particularly to calibrating magnetic-field based medical equipment having a plurality of magnetic field sources.

### BACKGROUND

Use of a magnetic field for medical interventions is becoming increasingly more prevalent. For instance, the following patent documents describe prior art medical applications harnessing a magnetic field: U.S. Pat. No. 9655539, U.S. Pat. No. 9381063, U.S. Pat. No. 9220425, U.S. Pat. No. 8986214, U.S. Pat. No. 8684010, U.S. Pat. No. 8457714, US20130006100, US20120310111, US20120289822, US20120288838, US20110092808, U.S. Pat. No. 7873401, US20100305402, US20090275828, US20090248014, US20050096589, etc.

Certain magnetic-field based systems use a magnetic field generated by a plurality of magnetic field sources for steering magnetotactic entities in a body of a subject.

Magnetotactic entities are defined as untethered entities where the source of propulsion or the system responsible for the displacement of the entity is part of, attached to, or embedded in the entity itself. Magnetotactic entities include a group of objects or microorganisms and any biological system or hybrid system including micro- and nano-systems or structures made of biological and/or synthetic (including chemical, artificial, etc.) materials and/or components where the directional motion can be influenced by inducing a torque from a directional magnetic field (e.g. from a permanent magnet) or electro-magnetic field (magnetic field includes here electro-magnetic field generated by an electrical current flowing in a conductor), a method referred to here as magnetotaxis where the direction of motion of such magnetotactic entities is influenced by a directional magnetic field (the magnetotactic entities can also be functionalized and be attached to other structures if required). These magnetotactic entities may be north-seeking, south-seeking, or a mixture of both. Examples of such magnetotactic entities include but are not limited to a single or a group (swarm, agglomeration, aggregate, etc.) of flagellated Magnetotactic Bacteria (MTB), or other bacteria or other microorganisms capable of self-propulsion and influenced for the purpose of directional control by a directional magnetic field that could have been modified previously accordingly from various methods including but not limited to cultivation parameters, genetics, or attached, embedded to other entities modified to allow control by magnetotaxis such as other cells (including red blood cells), or attached to a synthetic structure that can be influenced by a directional magnetic field or gradient, or by adding micro- or nano-components to the bacteria, cells, or other microorganisms to make the directional motion of the implementation including hybrid (made of biological and synthetic components) implementation sensitive to magnetotaxis or a directional magnetic field such as the one capable of influencing the direction of a magnetic nano-compass needle.

U.S. Patent No. 9,905,347, incorporated herein by reference, describes a system for steering magnetotactic entities in a subject. U.S. Patent No. 9,905,347 describes a system and method for generating a 3D-convergence point using at least three sets of magnetic field sources arranged along three axes or in three planes.

Moreover, International PCT patent application No. PCT/CA2021/051776, incorporated herein by reference, describes a system with six magnetic field sources positioned around a platform for receiving a subject, the system configured to guide magnetotactic entities in the subject.

However, the dimensions and configuration of these magnetic field-based systems make the adjustment of the poses of their magnetic field sources, during their installation and maintenance, challenging. A user manually adjusting the position and orientation of the magnetic field sources may unknowingly create errors when calibrating the positions and orientations of the magnetic field sources, these errors impacting the precision and accuracy of the magnetic field source system when treating a subject. Moreover, manually calibrating the pose of the magnetic field sources of the magnetic field source system is time-consuming.

### SUMMARY

The present disclosure relates to systems and methods for calibrating the pose of the magnetic field sources of a medical system with magnetic field sources to generate a magnetic field for the purposes of treating a subject.

The poses of the magnetic field sources, including their positions with respect to one another, are factored in when employing the medical system for selecting and defining the magnetic field sequences for steering or guiding magnetotactic entities in the body of a subject.

The present system includes position sensors for generating positional information that is analyzed to calculate the positions and orientations of the magnetic field sources in real-world of the magnetic field source system. The position sensors may be one or more cameras that capture the magnetic field sources in one or more image streams. The real-world poses of the magnetic field sources are compared to select poses of the magnetic field sources as defined in a three-dimensional model of the magnetic field sources, the magnetic field sources of the virtual model arranged with respect to a target isocenter for the magnetic field sources. The system generates instructions (e.g. provides visual cues on a display to move the magnetic field sources, provides guidance in the form of a string of characters appearing on a display, issues commands to actuate an automated change of position of the magnetic field sources) in order for the magnetic field sources to approach their select poses. As the poses of the magnetic field sources are gradually adjusted, further instructions may be provided by the system to further position the magnetics heads such that their poses approach that defined by the virtual model of the magnetic field sources.

The present disclosure further relates to a constellation of markers for affixing to the magnetic field sources, the constellation of markers captured by the position sensor(s) (e.g. cameras). The positions of the markers of the constellation of markers in image space is used to calculate the poses of each of the magnetic field sources in the real-world. Each of the constellation of markers includes at least three markers joined to a support frame for three-dimension determination. The support frame may include a mount for affixing the constellation of markers to a magnetic field source (e.g. a tip of a magnetic field source).

A broad aspect is a method for guiding the positioning of magnetic field sources of a magnetic-field based system, the system comprising a plurality of magnetic field sources. The method includes receiving positional data for each of the plurality of magnetic field sources; determining a pose of each magnetic field source of the plurality of magnetic field sources from the positional and orientation data; generating a computer model of a target layout of the plurality of magnetic field sources, defining a select pose for each of the plurality of magnetic field sources; and for each magnetic field source of the plurality of magnetic field sources, comparing the select pose with the determined pose to calculate one or more of a rotation and translation of the magnetic field source; and generating instructions to adjust one or more of a position and orientation of at least one of the plurality of the magnetic field sources from the calculated one or more of a rotation and translation, for causing the plurality of magnetic field sources to approach the target layout of the computer model.

In some embodiments, the positional data for each of the plurality of magnetic field sources may be generated from one or more cameras, wherein a combined field of view of the one or more cameras encompasses each of the plurality of magnetic field sources.

In some embodiments, each magnetic field source of the plurality of magnetic field sources may include a constellation of markers attached thereto, and wherein the positional data may be further generated from the constellation of markers captured by the one or more cameras.

In some embodiments, supporting frames may be joined to the plurality of magnetic field sources, each constellation of markers may be joined to a supporting frame of the supporting frames configured to position at least three markers of the constellation of markers to provide information for defining the pose of each magnetic field source of the plurality of magnetic field sources, where each supporting frame of the supporting frames may be joined to a different magnetic field source of the plurality of magnetic field sources.

In some embodiments, one or more of the supporting frames may have a different shape from other supporting frames of the one or more supporting frames.

In some embodiments, the supporting frames may be two dimensional.

In some embodiments, the positional data for each of the plurality of magnetic field sources may be generated from a robotic arm that is positioned to contact one or more of the plurality of magnetic field sources.

In some embodiments, the plurality of magnetic field sources may include three pairs of magnetic field sources.

In some embodiments, the target layout may define a position of the plurality of magnetic field sources, wherein each pair of the three pairs may be orthogonal with respect to each of the other two pairs of the three pairs.

In some embodiments, the method may include receiving further positional data for each of the plurality of magnetic field sources following an adjustment of the one or more of a position and orientation of the magnetic field source in accordance with the instructions; determining an updated pose of each magnetic field source of the plurality of magnetic field sources from the positional data; and for each magnetic field source of the plurality of magnetic field sources, comparing the select pose with the determined updated pose to calculate one or more of a further rotation and further translation of the magnetic field source; and generating instructions to adjust one or more of the position and the orientation of at least one of the plurality of magnetic field sources from the corresponding calculated one or more of an additional rotation and additional translation, for causing the plurality of magnetic field sources to further approach the target layout.

In some embodiments, prior to the comparing, the method may include adjusting an orientation of the computer model of a target layout to align a computer magnetic field source of the model with a pose of the corresponding magnetic field source of the plurality of magnetic field sources.

Another broad aspect is a computing device for guiding the positioning of magnetic field sources of a magnetic-field based system, the system includes a plurality of magnetic field sources, the computing device comprising a processor; and memory storing program code that, when executed by the processor, causes the processor to receive positional data for each of the plurality of magnetic field sources; determine a pose of each magnetic field source of the plurality of magnetic field sources from the positional data; generate a computer model of a target layout of the plurality of magnetic field sources, defining a select pose for each of the plurality of magnetic field sources; and for each magnetic field source of the plurality of magnetic field sources, compare the select pose with the determined pose to calculate one or more of a rotation and translation of the magnetic field source; and generate instructions to adjust one or more of a position and orientation of at least one of the plurality of the magnetic field sources from the calculated one or more of a rotation and translation, for causing the plurality of magnetic field sources to approach the target layout.

In some embodiments, the positional data for each of the plurality of magnetic field sources may be generated from one or more cameras, wherein a combined field of view of the one or more cameras may encompass each of the plurality of magnetic field sources.

In some embodiments, each magnetic field source of the plurality of magnetic field sources may include a constellation of markers attached thereto, and wherein the positional data may be further generated from the constellation of markers captured by the one or more cameras.

In some embodiments, supporting frames may be joined to the plurality of magnetic field sources, each constellation of markers may be joined to a supporting frame of the supporting frames configured to position at least three markers of the constellation of markers to provide information for defining the pose of each magnetic field source of the plurality of magnetic field sources, where each supporting frame of the supporting frames may be joined to a different magnetic field source of the plurality of magnetic field sources.

In some embodiments, one or more of the supporting frames may have a different shape from other supporting frames of the one or more supporting frames.

In some embodiments, the supporting frames (substrates) may be two dimensional.

In some embodiments, the positional data for each of the plurality of magnetic field sources may be generated from a robotic arm that is positioned to contact one or more of the plurality of magnetic field sources.

In some embodiments, the plurality of magnetic field sources may include three pairs of magnetic field sources.

In some embodiments, the target layout may define a position of the plurality of magnetic field sources, wherein each pair of the three pairs may be orthogonal with respect to each of the other two pairs of the three pairs.

In some embodiments, the program code, when executed by the processor, may further cause the processor to receive further positional data for each of the plurality of magnetic field sources following an adjustment of the one or more of a position and orientation of the magnetic field source in accordance with the instructions; determine an updated pose of each magnetic field source of the plurality of magnetic field sources from the positional data; and for each magnetic field source of the plurality of magnetic field sources, compare the select pose with the determined updated pose to calculate one or more of a further rotation and further translation of the magnetic field source; and generate instructions to adjust one or more of the position and the orientation of at least one of the plurality of magnetic field sources from the corresponding calculated one or more of an additional rotation and additional translation, for causing the plurality of magnetic field sources to further approach the target layout.

In some embodiments, prior to the comparing, wherein the program code, when executed by the processor, may further cause the processor to adjust an orientation of the computer model of a target layout to align a computer magnetic field source of the model with a pose of the corresponding magnetic field source of the plurality of magnetic field sources.

Another broad aspect is a non-transitory computer-readable medium having stored thereon program instructions for guiding the positioning of magnetic field sources of a magnetic-field based system, the program instructions executed by a processing unit for receiving positional data for each of the plurality of magnetic field sources; determining a pose of each magnetic field source of the plurality of magnetic field sources from the positional data; generating a computer model of a target layout of the plurality of magnetic field sources, defining a select pose for each of the plurality of magnetic field sources; and for each magnetic field source of the plurality of magnetic field sources, comparing the select pose with the determined pose to calculate one or more of a rotation and translation of the magnetic field source; and generating instructions to adjust one or more of a position and orientation of at least one of the plurality of the magnetic field sources from the calculated one or more of a rotation and translation, for causing the plurality of magnetic field sources to approach the target layout.

In some embodiments, the positional data for each of the plurality of magnetic field sources may be generated from one or more cameras, wherein a combined field of view of the one or more cameras may encompass each of the plurality of magnetic field sources.

In some embodiments, each magnetic field source of the plurality of magnetic field sources may include a constellation of markers attached thereto, and wherein the positional data may be further generated from the constellation of markers captured by the one or more cameras.

In some embodiments, supporting frames may be joined to the plurality of magnetic field sources, each constellation of markers may be joined to a supporting frame of the supporting frames configured to position at least three markers of the constellation of markers to provide information for defining the pose of each magnetic field source of the plurality of magnetic field sources, where each supporting frame of the supporting frames may be joined to a different magnetic field source of the plurality of magnetic field sources.

In some embodiments, one or more of the supporting frames may have a different shape from other supporting frames of the one or more supporting frames.

In some embodiments, the supporting frames (substrates) may be two dimensional.

In some embodiments, the positional data for each of the plurality of magnetic field sources may be generated from a robotic arm that is positioned to contact one or more of the plurality of magnetic field sources.

In some embodiments, the plurality of magnetic field sources may include three pairs of magnetic field sources.

In some embodiments, the target layout may define a position of the plurality of magnetic field sources, wherein each pair of the three pairs may be orthogonal with respect to each of the other two pairs of the three pairs.

In some embodiments, the program instructions may be further executed by the processing unit for receiving further positional data for each of the plurality of magnetic field sources following an adjustment of the one or more of a position and orientation of the magnetic field source in accordance with the instructions; determining an updated pose of each magnetic field source of the plurality of magnetic field sources from the positional data; and for each magnetic field source of the plurality of magnetic field sources, comparing the select pose with the determined updated pose to calculate one or more of a further rotation and further translation of the magnetic field source; and generate instructions to adjust one or more of the position and the orientation of at least one of the plurality of magnetic field sources from the corresponding calculated one or more of an additional rotation and additional translation, for causing the plurality of magnetic field sources to further approach the target layout.

In some embodiments, prior to the comparing, wherein the program instructions may be further executed by the processing unit for adjusting an orientation of the computer model of a target layout to align a computer magnetic field source of the model with a pose of the corresponding magnetic field source of the plurality of magnetic field sources.

### BRIEF DESCRIPTION OF THE DRAWINGS

The invention will be better understood by way of the following detailed description of embodiments of the invention with reference to the appended drawings, in which:

FIG. 1 is a block diagram of an exemplary system for calibrating poses of magnetic field sources of a magnetic field-based medical system, exemplary position sensors and an exemplary a magnetic field-based medical system;

FIG. 2 is a drawing of a perspective view of an exemplary a magnetic field-based medical system with six magnetic field sources;

FIG. 3 is a block diagram of an exemplary software architecture for position guidance of magnetic field sources of a magnetic field-based medical system;

FIG. 4 is a drawing of an exemplary set of constellations of markers affixed to magnetic field sources of an exemplary magnetic field-based medical system;

FIG. 5 is a drawing of close-up version of the exemplary set of constellations of markers affixed to the magnetic field sources of the exemplary magnetic field-based medical system of FIG. 4;

FIG. 6 is a drawing of an exemplary constellation of markers including 6 markers; and

FIG. 7 is a flowchart diagram of an exemplary method for calibrating the poses of magnetic field sources of a magnetic field-based medical system.

### DETAILED DESCRIPTION

The present disclosure describes systems and methods for calibrating poses of magnetic field sources of a magnetic field-based medical system (also referred to herein as a magnetic field source system) for treating subjects using the magnetic field, namely by guiding or steering magnetotactic entities in a body of the subject using the magnetic field generated by the magnetic field sources.

The systems and methods receive positional data on the magnetic field sources that is generated by position sensors (e.g. one or more cameras capturing each of the magnetic field sources). The positional data is then analyzed to determine a pose for each of the magnetic field sources of the magnetic field source system.

A virtual three-dimensional model of the magnetic field sources of the medical system is generated, representing an ideal or select pose for each of the magnetic field sources of the magnetic field source system with respect to a defined isocenter for the magnetic field sources.

Each of the poses of the magnetic field sources of the virtual model are compared to their respective poses of the magnetic field sources in the real-world. Recommendations are generated following the comparison to adjust the poses of one or more of the magnetic field sources in the real-world, in order for these poses to approach that of their virtual counterparts as defined in the virtual three-dimensional model of the magnetic field sources. Recommendations may be displayed as visual cues on a screen (through imagery showing the difference in poses between the real-world magnetic field sources and the magnetic field sources of the virtual models; through a string of characters providing instructions on how to move the magnetic field sources appearing on a display; through instructions communicated by sound through one or more speakers; by causing an actuation of the movement (e.g. mechanically, pneumatically, etc.) of the real-world magnetic field sources, causing the real-world magnetic field sources to approach the positions represented in the three-dimensional virtual model, etc.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

### DEFINITIONS:

In the present disclosure, by “medical intervention,” it a meant a procedure performed on a subject for at least one of treatment (e.g., removal of a mass – tumor – through surgery), imaging (e.g., endoscopy, colonoscopy, optical coherence tomography, etc.) and diagnosis (which can involve one or more imaging techniques).

In the present disclosure, by “subject”, it is meant mammals and non-mammals. Mammals mean any member of the mammalia class including, but not limited to, humans. Non-mammals include birds, reptiles, etc. The term “subject” should not bring on any limitations as to the sex or age. Even though the configuration of the system has been determined to accommodate a human subject adopting different intervention positions as explained herein, it will be understood that the present system can be used with non-human subjects (i.e. animals), without departing from the present teachings, provided that the animal can fit within the space defined by the structure of the system and there is sufficient room for the members of the medical staff (e.g. veterinarian; technician) to access the subject and/or circulate around the subject.

In the present disclosure, by “isocenter”, it is meant, for a magnetic field source system, the point for the magnetic field source system where all of the axes of the magnetic field sources, when the magnetic field sources are coils, intersect.

### EXEMPLARY SYSTEM FOR CALIBRATING POSES OF MAGNETIC FIELD SOURCES OF A MAGNETIC FIELD-BASED MEDICAL SYSTEM:

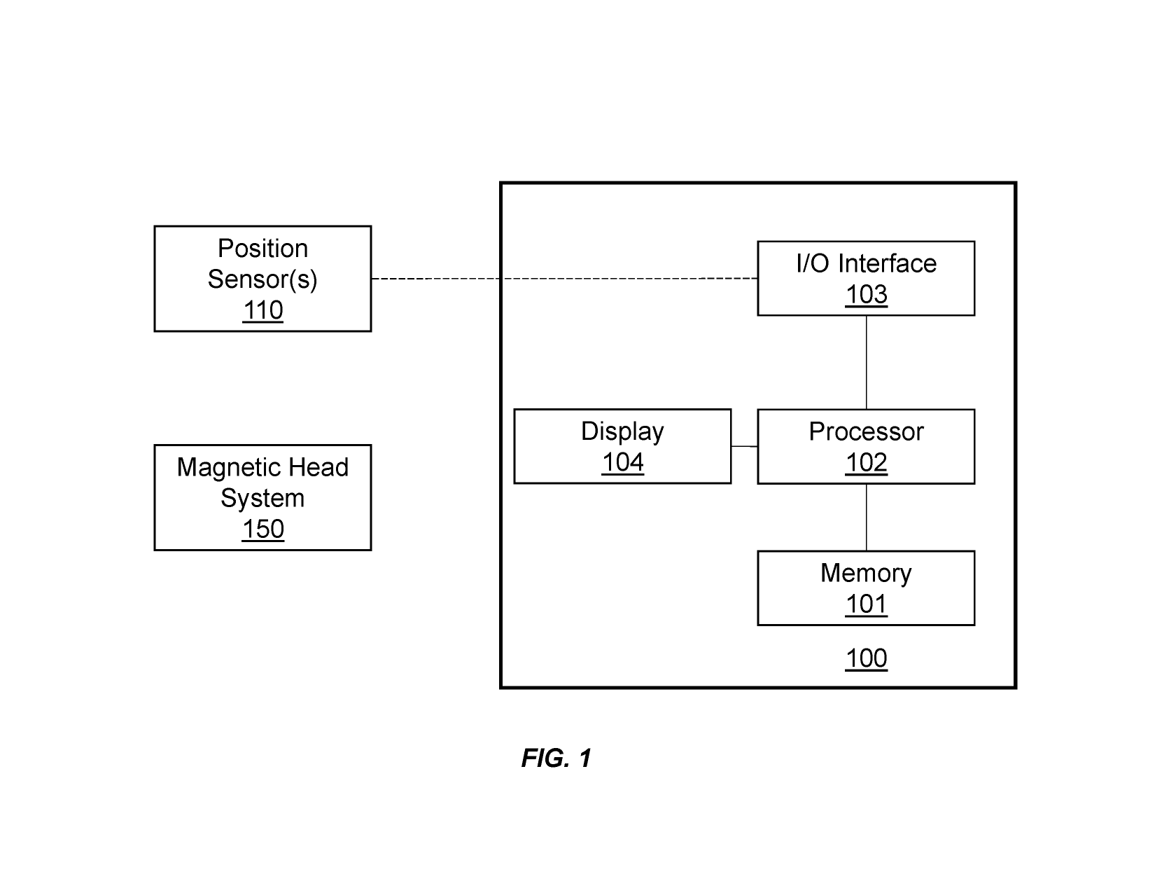

Reference is now made to FIG. 1, illustrating an exemplary system 100 for calibrating poses of the magnetic field sources of a magnetic field source system 150, the magnetic field source system 150 including magnetic field sources for generating a magnetic field. The magnetic field may be used for guiding or steering magnetotactic entities in a body of a subject for a medical intervention (treatment, diagnostics and/or imagery). In some instances, the system 100 may be part of the magnetic field source system 150 (e.g. system 100 may implemented or integrated into a computer for using the magnetic field source system 150).

The system 100 has at least one processor 102, memory 101 and at least one input/output interface 103 for communication with the one or more position sensors 110 and/or the magnetic field source system 150. The system 100 may include a display 104.

The system 100 may have an interface 103 for each of the position sensors 110. A separate I/O interface 103 may also be provided in system 100 for communicating with the I/O interface of the magnetic field source system 150.

The processor 102 may be a general-purpose programmable processor. In this example, the processor 102 is shown as being unitary, but the processor 102 may also be multicore, or distributed (e.g. a multi-processor).

The computer readable memory 101 stores program instructions and data used by the processor 102. The computer readable memory 101 may also store magnetic field source virtual models, poses or real-world magnetic field sources, etc. The memory 101 may be non-transitory. The computer readable memory 101, though shown as unitary for simplicity in the present example, may comprise multiple memory modules and/or caching. In particular, it may comprise several layers of memory such as a hard drive, external drive (e.g. SD card storage) or the like and a faster and smaller RAM module. The RAM module may store data and/or program code currently being, recently being or soon to be processed by the processor 102 as well as cache data and/or program code from a hard drive. A hard drive may store program code and be accessed to retrieve such code for execution by the processor 102 and may be accessed by the processor 102 to store and access data. The memory 101 may have a recycling architecture for storing, for instance, positional data related to the magnetic field sources, poses values for the real-world magnetic field sources, etc. where older data files are deleted when the memory 101 is full or near being full, or after the older data files have been stored in memory 101 for a certain time.

The I/O interface 103 is in communication with the processor 102.

The processor 102, the memory 101 and the I/O interfaces 103 may be linked via bus connections.

The display 104 may be a screen (e.g. a touchscreen), a virtual reality headset, etc. for displaying information to a user that is generated by or related to the system 100. The virtual three-dimensional model of the magnetic field sources may be displayed on the display 104 and compared to the real-world poses of the corresponding magnetic field sources of the magnetic field source system 150. Instructions for adjusting the real-world poses of the magnetic field sources may also be presented on the display 104.

In some instances, system 100 may include one or more speakers (not shown) for sharing by audio for sharing the instructions for adjusting the real-world poses of the magnetic field sources.

In some instances, the system 100 may include a user input interface (not shown), for receiving user input, e.g., for initiating the calibration process of the magnetic field sources, adjusting the real-world poses of one or more of the magnetic field sources, etc. In some instances, the user input interface may be one or more of a keyboard, a touchscreen, a mouse, a trackpad, a movement sensor, a microphone, a touchscreen, etc.

The one or more position sensors 110 are in communication with the I/O interface 103 of the system 100, to transmit to the system 100 the positional information of the magnetic field sources of the magnetic field source system 150 generated by the one or more position sensors 110. The one or more position sensors 110 may be RGB (red-green-blue) camera(s), infrared camera(s), a surface laser, a light detection and ranging (LIDAR) scanner, a time-of-flight camera, an end effector of a robotic arm (where changes in the pose of the robotic arm can be correlated to the pose, and change of pose, of the magnetic field source), etc. In one embodiment, the one or more position sensors 110 are RGB cameras, having their collective field of views covering the magnetic field sources of the magnetic field source system 150. In some instances, the entire bodies of the magnetic field sources are captured by the one or more RGB cameras.

The system 100 may be a computing device (e.g. a desktop computer, a laptop computer, a tablet computer, etc.)

### EXEMPLARY MAGNETIC FIELD SOURCE SYSTEM CONFIGURED TO GENERATE A MAGNETIC FIELD FOR STEERING MAGNETOTACTIC ENTITIES:

The magnetic field source system 150 includes two or more magnetic field sources that surround a platform adapted to receive a subject.

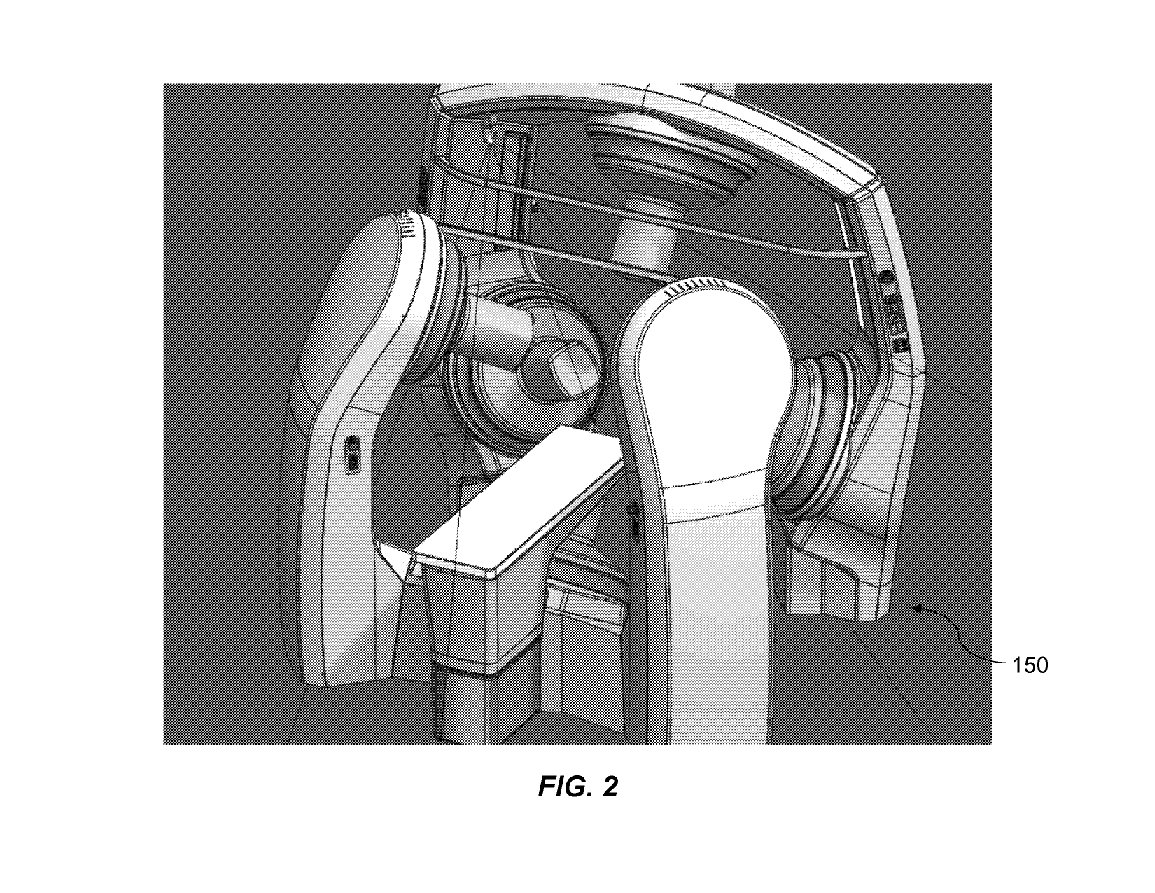

An exemplary magnetic field source system 150 is illustrated at FIG. 2. The example of FIG. 2 shows six magnetic field sources or three pairs of two magnetic field sources. Each pair of the three pairs is aligned with respect to one of three axes (x, y, z).

However, it will be understood that the number of magnetic field sources of the magnetic field source system 150 may vary depending on the structure of the magnetic field source system 150 and its purpose (e.g. for assisting the magnetotactic entities to navigate along all three axes x, y and z; or one axis, etc.) For instance, in some examples, the magnetic field source system 150 may include two magnetic field sources, four magnetic field sources, seven magnetic field sources, eight magnetic field sources, etc.

In the example of FIG. 2, the system 150 includes a platform for receiving the subject, six magnetic field sources grouped in pairs, and a support structure for supporting the magnetic field sources in a given configuration.

The system 150 may include an imaging device such as an X-ray imaging device such as a C-arm.

The magnetic field sources generate the magnetic field for steering the magnetotactic entities. As such, the magnetic field sources each have one or more magnetic coils surrounding a ferromagnetic core that generates a magnetic field when a current is passed therethrough. In this example, the magnetic field sources are paired together, thereby forming at least three pairs. Each of the pairs of magnetic field sources is defined by one axis of three axes that are orthogonal with respect to one another. In some embodiments, none of the three axes that define the orientation of the magnetic field sources is parallel with the floor, and none of the three axes is parallel with any of reference axes x, y and z (where the x-axis is parallel with the floor). The pairs of magnetic field sources may establish a point in a body of the subject where the magnetotactic entities will navigate towards and converge, using, for instance, the techniques described in U.S. 9,905,347. However, a support structure may be needed to accommodate the size of the magnetic field sources and to meet the requirement of providing sufficient space to fit a subject amongst the magnetic field sources such that the convergence point or magnetic field generated by the magnetic field sources can be generated in the body of the subject, and to provide sufficient space to allow a physician to access the subject when lying on the platform 105 (considering that the space occupied by the subject and physician may depend on the nature of the procedure to be performed on the subject, as explained herein).

Support structure supports each of the magnetic field sources in their given configuration where the magnetic field sources of each pair of magnetic field sources are positioned in opposition to one another along an axis oriented orthogonally with respect to the axes of the other pairs of magnetic field sources for generating a magnetic field capable of steering the magnetotactic entities in three dimensions. The magnetic field sources of each pair of magnetic field sources are facing one another. The support structure may include a support arch and two support arms.

The support arch and the two support arms may be positioned at or near opposite ends of the platform such that the magnetic field sources supported thereby can be fixed in a proper orientation for generating the necessary magnetic field adapted for creating a torque in each of the three axes x, y and z on magnetotactic entities.

Each of the support arms may support one magnetic field source of a different pair of magnetic field sources. The magnetic field source may be positioned at or near a top of the support arm. The support arm may be shaped such that the support arm is arched downward, or may have a vertical portion and a portion for receiving a magnetic field source that is at an angle with the vertical portion of the support arm, thereby orienting the magnetic field source at least slightly downward, towards the platform. As such, the shape of the support may be determined to position the magnetic field source attached thereto at a proper angle to face the corresponding other magnetic field source of the pair of magnetic field sources. The orientation of the magnetic field source may be such that the magnetic field source supported by the support arm is facing the corresponding magnetic field source of the magnetic field source pair, as described herein. In some embodiments, each of the support arms may be connected to one another at a base portion (e.g. joins the base of the platform). In other embodiments, each of the support arms may be separate from the platform.

In some examples, the support arms may be configured to move away and towards the platform, e.g. in order to create additional space for placing the patient on the platform. For instance, the support arms may be positioned on rails, where the support arms may slide along the rails to create additional space.

In some examples, the magnetic field sources may be pivotably attached to the support arms or the support arch, such that they may be displaced in order to further create space between the magnetic field sources, e.g. for placing the patient on the platform.

The support arch may support one or more magnetic field sources. As is shown in the example of FIG. 2, the support arch supports three magnetic field sources. A first magnetic field source supported by the support arch is paired with a magnetic field source supported by the first support arm, a second magnetic field source supported by the support arch is paired with a magnetic field source supported by a second support arm, and a third magnetic field source supported by the supper arch, centered on the support arch, is paired with the magnetic field source positioned under the platform.

The support arch has two leg portions, contacting the ground and that are substantially vertical (the leg portion may have a slight curve or bend as illustrated, for instance, in FIG. 2) and an arch portion that is curved, interconnecting the two leg portions of the support arch.

Each of the leg portions of the support arch may receive one magnetic field source. The magnetic field source may be located near or at the bottom of the leg portion of the support arch. The width or thickness of the leg portion of the support arch may be greater towards the bottom in order to support the magnetic field source (e.g. may not be uniform). In some examples, the leg portion of the support arch may include a user input interface for controlling the system (e.g. the generating of a magnetic field using the pairs of magnetic field sources).

The arch portion of the support arch creates a space as a result of the elevation of the middle of the arch for an intervening physician to access the subject, as shown in FIG. 2.

In some examples, instead of a pair of support arms, the system 150 may include a second support arch for receiving the two magnetic field sources instead of the support arms (not shown).

It will be understood that the orientation and position of the magnetic field sources 102 shown in FIG. 2 illustrate one example of same that was developed for meeting size constraints and to generate sufficient space for the subject and intervening members of medical staff to perform certain medical interventions, as described herein, where certain medical interventions require that the subject be laid in a given position.

The platform is adapted to receive the subject into whom will be introduced the magnetotactic entities. As such, the length of the platform may be sufficient to receive one of possible subjects of different heights and widths lying down (e.g. a subject with a height that is less than 6.5 feet). The platform may be adapted to move back-and-forth, side-to-side and/or up-and-down (i.e. along one or more of the three axes x, y, and z). In some embodiments, the platform may also be configured to rotate clockwise and/or counter-clockwise. The movement of the platform may be controlled by a user using a user input interface, such as the one appearing on the leg portion of the support arch in FIG. 2. In other embodiments, the system 150 may be controlled remotely (e.g. using a remote control that communicates wirelessly with the system 150; a computing device such as a platform computer or smartphone including program code for controlling the system 150 when executed by the processor of the computing device, etc.)

The platform may be layered with a thin mattress or cushion to provide comfort for the subject. In some examples, the platform may have extensions for receiving the arms of the subject.

The imaging device is configured to obtain information on the anatomy of the subject (imaging of the inside of the subject). In some embodiments, this information may be used to monitor the progress of the magnetotactic entities in the subject, determine the location of the target site towards which the magnetotactic entities will be steered, to provide visuals for the purpose of a medical intervention such as a surgery, etc. The imaging device may be an X-ray image intensifier, such as a C-arm. However, it will be understood that other imaging devices may be used without departing from the present teachings.

In some examples, as shown in FIG. 2, the imaging device may fit in a space between the support arm and the support arch. When the imaging device is a C-arm, the arch of the C-arm may arch around the platform such that the C-arm can gather information on the subject lying on the platform, where, e.g., the platform may move up and down with respect to the C-arm such that the C-arm can provide imaging data along a length of a body of the subject. In some embodiments, the imaging device may be displaced manually or remotely, having, for instance, a pair of wheels for rolling the imaging device into different positions.

The magnetic field sources may have a range of motion for adjustment, where their orientation and position may be adjusted with respect to the support structure of the magnetic field source system 150. Each of the magnetic field sources of the system 150 may include an actuator for adjusting the pose of the magnetic field source as a function of instructions received from the system 100, via the I/O interface 103. However, it will be understood that in some situations, the range of motion of each of the magnetic field sources may be limited, e.g., due to how the magnetic field source is connected to the support structure of the magnetic field source system 150. The limitations in the range of motion of the magnetic field sources may be considered by the system 100 when calculating the instructions to change the pose of the magnetic field sources, in order for the poses of the real-world magnetic field sources to match the select poses of the magnetic field sources of the virtual model.

### EXEMPLARY POSITION GUIDANCE SOFTWARE ARCHITECTURE:

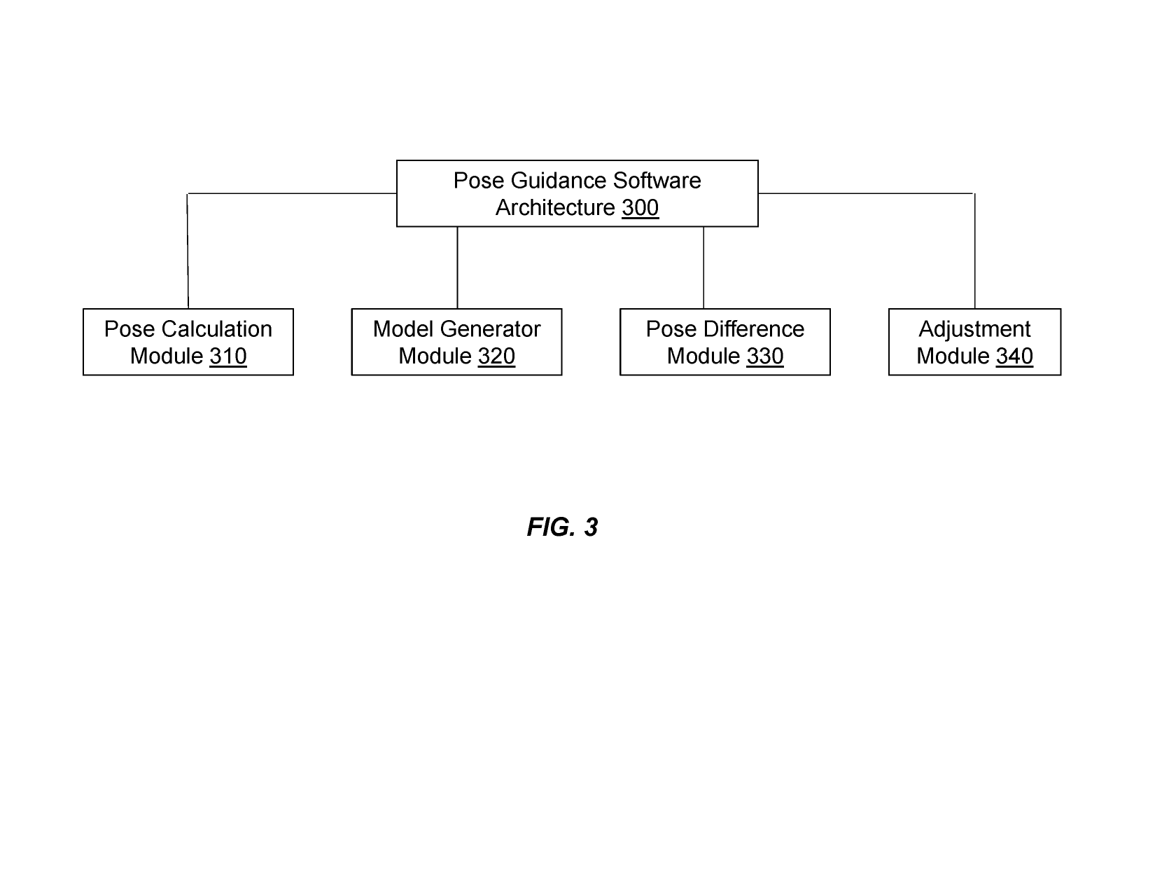

Reference is now made to FIG. 3, illustrating an exemplary pose guidance software architecture 300 of the system 100 for adjusting the pose of the magnetic field sources of a magnetic field source system 150.

The system 100 has program code, stored in memory 101, that includes a pose calculation module 310. The system 100 has program code, stored in memory 101, that includes a model generator module 320. The system 100 has program code, stored in memory 101, that includes a pose difference module 330. The system 100 has program code, stored in memory 101, that includes an adjustment module 340. Each of the pose calculation module 310, the model generator module 320, pose difference module 330 and/or adjustment module 340 includes program code configured to implement the functionality of the modules as is described herein.

The pose calculation module 310 includes program code stored in memory 101 that, when executed by the processor 102, causes the processor 102 to receive positional data from the one or more position sensors 110 related to each of the magnetic field sources. The pose calculation module 310 further causes the processor 102 to calculation a position and orientation (i.e. a pose) from the positional data for each of the magnetic field sources. The pose calculation module 310 may cause the processor 102 to calculate the pose for a magnetic field source by performing image recognition of the magnetic field source in an image stream and comparing the object of the magnetic image in the image space to one or more representations of a magnetic field source with a known pose, and/or by performing object tracking of one or more markers in an image stream related to the pose of the magnetic field source. The pose of the magnetic field source may be calculated after determining the relative position of each of the markers with respect to one another.

The model generator module 320 includes program code stored in memory 101 that, when executed by the processor 102, causes the processor 102 to generate a virtual three-dimensional model of magnetic field sources that are representative of the magnetic field sources of the magnetic field source system 150. The virtual three-dimensional model may also define an isocenter for the magnetic field sources of the virtual model. The model generator module 320 may also cause the processor 102 to render the virtual three-dimensional model of the magnetic field sources on the display 104 of the system 100.

The pose difference module 330 includes program code stored in memory 101 that, when executed by the processor 102, causes the processor 102 to measure a difference in the pose between the real-world magnetic field sources and the select pose of corresponding magnetic field sources represented in the virtual three-dimensional model of the magnetic field sources of the magnetic field source system 150. The pose difference module 330 may cause the processor 102 to map the pose of each of the real-world magnetic field sources to their corresponding virtual magnetic field source in the virtual model by, e.g., for each of the poses of the real-world magnetic field sources, determining which pose of the real-world magnetic field sources is closest to a pose of one of the magnetic field sources of the virtual models (the mapping may be performed for each of the poses of the real-world magnetic field sources).

In some instances, the pose difference module 330 may cause the processor 102 to render a virtual representation of the real-world magnetic field sources, their representation determined from their respective calculated poses, on a display 104, e.g., next to or overlapping with the rendered virtual model of the magnetic field sources.

The adjustment module 340 includes program code stored in memory 101 that, when executed by the processor 102, causes the processor 102 to calculate, for one or more of the magnetic field sources (in some instances, for each of the magnetic field sources), from the calculated pose of the magnetic field source, a rotation and/or a translation for causing the real-world magnetic field source to adjust its pose such that the pose of the real-world magnetic field source approaches (e.g. matches) the pose of its corresponding virtual magnetic field source.

The adjustment module 340 further causes the processor 102 to generate the instructions to adjust the pose of the real-world magnetic field source(s). The instructions may be displayed on the display 104 (e.g. as a string of characters, through visual representations guiding a user in adjusting the pose of the magnetic field source, etc.), shared through a speaker with a user, transmitted to one or more actuators for causing an adjustment of the pose of one or more of the magnetic field sources, etc.

In some instances, where the range of motion of one or more of the magnetic field sources is insufficient for enabling each of the real-world magnetic field sources of the magnetic system 150 to match the pose of the corresponding magnetic field source in the virtual model, the adjustment module 340 may cause the processor 102 to adjust an orientation and/or position of the virtual model of the magnetic field sources, to account for a magnetic field source with a pose sufficiently different from that of its corresponding real-world magnetic field source and where the range of motion of the real-world magnetic field source is insufficient to result in the pose of that magnetic field source matching the select pose of the magnetic field source, as defined in the corresponding virtual model.

In some instances, following adjustment to the pose of one or more of the real-world magnetic field sources, the adjustment module 340 may further cause the processor 102 to call the pose calculation module 310 to further determine the pose of the real-world magnetic field sources. Each of the pose calculation module 310, the model generator module 320 and pose difference module 330 may then operate sequentially to determine if the real-world poses of the magnetic field sources match the select poses of the magnetic field sources defined by the virtual model of the magnetic field sources. If not, the adjustment module 340 may further cause the processor 102 to generate further instructions to cause an additional adjustment of the pose of one or more of the real-world magnetic field sources. The process of sequentially calling each of the pose calculation module 310, the model generator module 320, the pose difference module 330 and the adjustment module 340 may be repeated until the poses of the real-world magnetic field sources match the select poses of the magnetic field sources, as defined in the virtual model of the magnetic field sources.

### EXEMPLARY CONSTELLATION OF MARKERS USE FOR IDENTIFYING A POSE OF A REAL-WORLD MAGNETIC FIELD SOURCE:

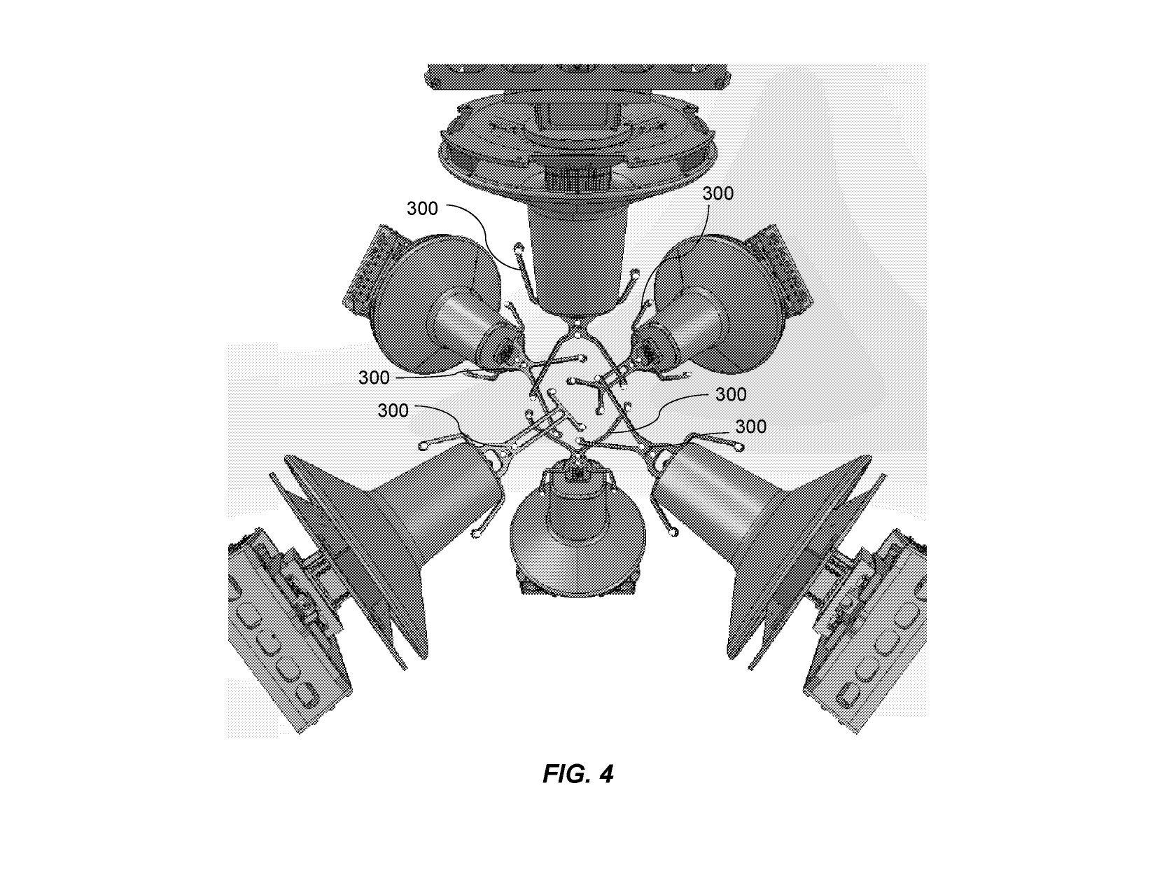

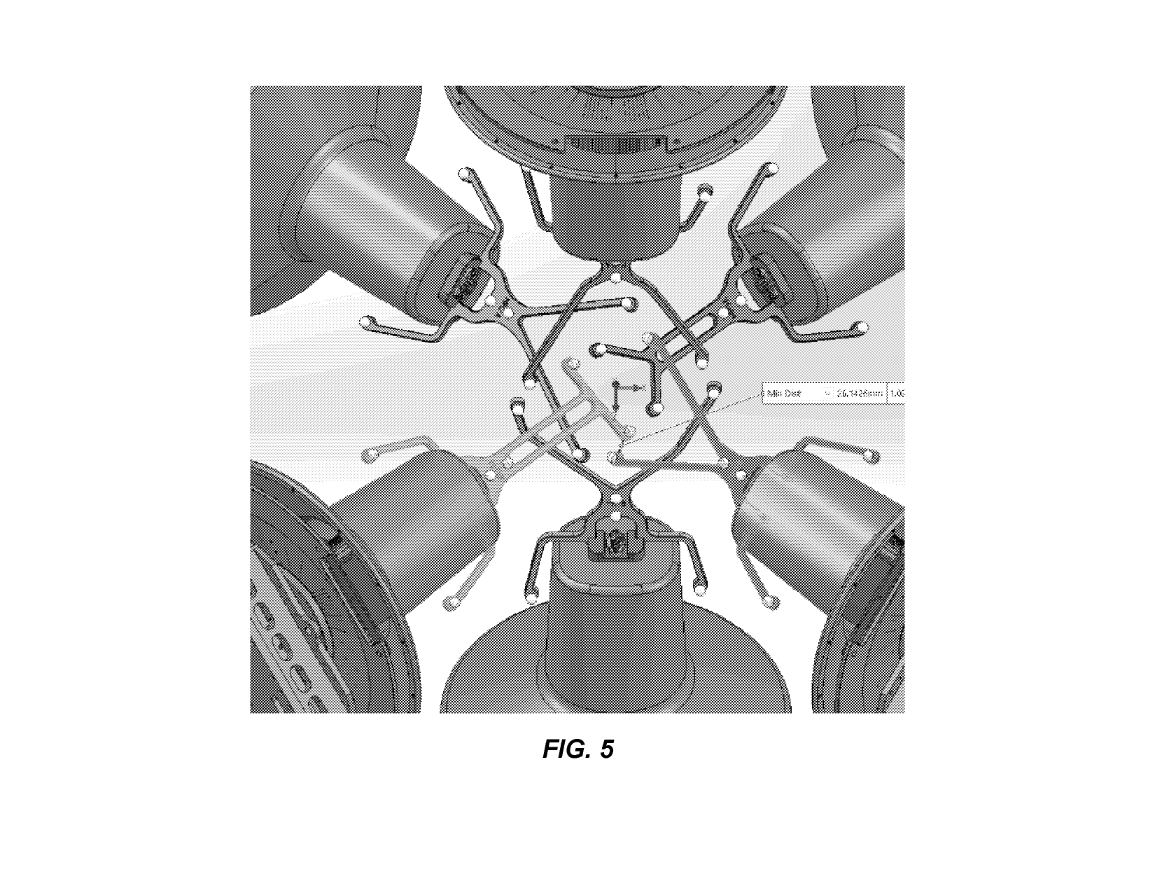

Reference is now made to FIGS. 4 and 5, illustrating a plurality of constellations of markers 300 for use in identifying a pose of real-world magnetic field sources of a magnetic field source system (e.g. magnetic field source system 150).

Each of the constellation of markers 300 is affixed to a magnetic field source (e.g. a tip of a magnetic field source as shown in FIG. 5).

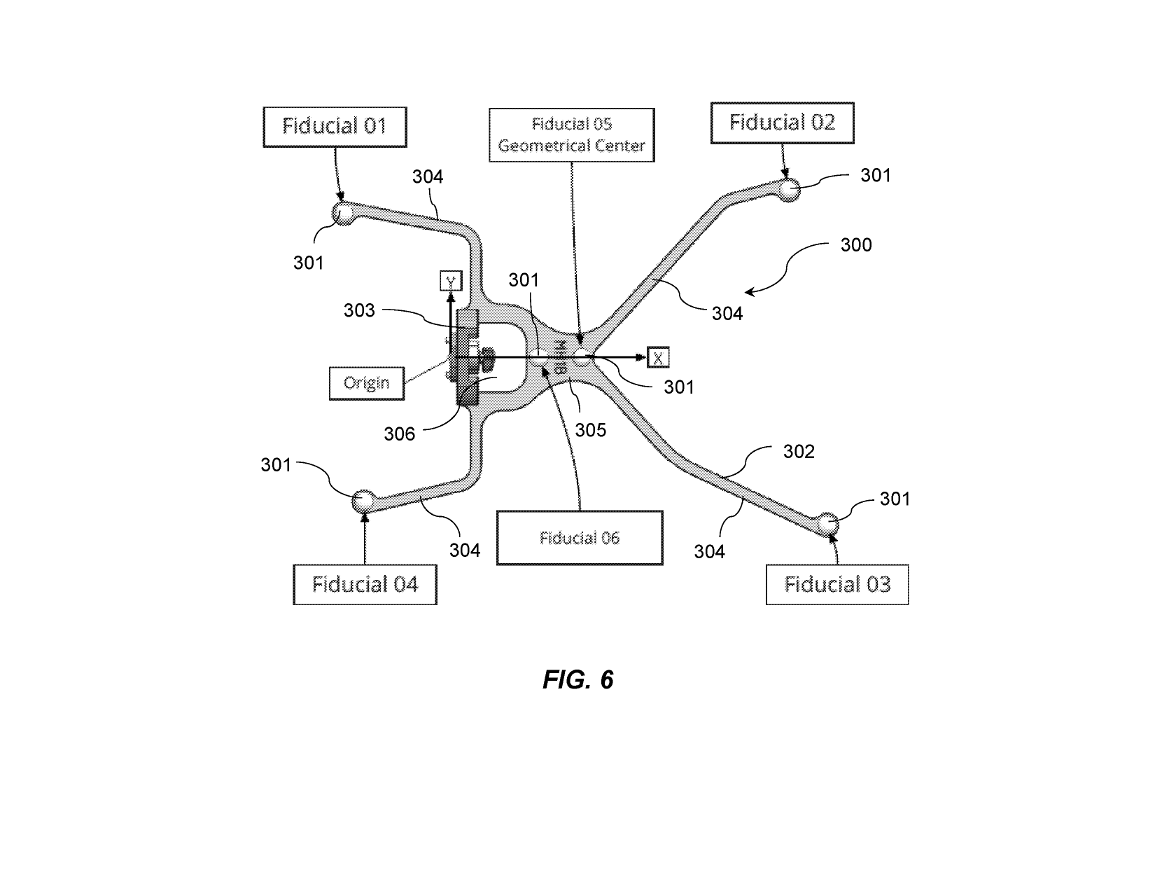

Reference is made to FIG. 6, illustrating an exemplary constellation of markers 300 for use in identifying a pose of a magnetic field source. The constellation of markers 300 includes at least three markers 301 (6 markers or fiducials 301 in the example of FIG. 6), a frame 302 and a mount 303.

The markers 301 are joined to the frame 302. In one embodiment, the frame 302 may be made out of aluminum. The markers 301 may be made from plastic (e.g. plastic balls) that are coated with a reflective tape or a reflective paint.

A mount 303 is further joined to the frame 302, the mount 303 adapted to join the constellation of markers 300 to a magnetic field source.

The frame 302 includes appendages 304 extending away from a main body 305. In some instances, a marker 301 may be joined to an end of each of the appendages 304. In some instances, one or more (e.g. two) markers 301 may be located at the main body 305 of the frame 302. The markers 301 located on the main body 305 of the frame 302 may be aligned with the mount 303. In some embodiments, the frame may be made out of plastic.

A space may be defined between the mount 303 and the main body 305 of the frame 302.

A set of constellations of markers 300 may be provided, where each constellation of markers of the set of constellations of markers 300 may be for affixing to a different magnetic field source of the magnetic field source system 150.

Due to spatial limitations between the magnetic field sources of the magnetic field source system 150, the frames 302 of the constellations of markers 300 of the set of constellations of markers 300 may have different shapes. This difference in shapes between the frames 302 of the constellations of markers 300 of the set of constellations of markers may be to enable the placement of the markers 301 at the ends of the appendages 304 without the different appendages 304 of the constellations of markers 300 contacting one another, and over the range of positions that may be adopted by the magnetic field sources. Moreover, the difference in shape of the frames 302 of the constellations of markers 300 may also create space between markers 301 of different constellations of markers 300 of the set of constellations of markers, such that the markers 301 of the different constellations of markers 300 may be distinguished from one another when captured by the one or more position sensors 110 (e.g. on one or more image stream, to limit obstruction of the markers by neighbouring constellations of markers).

It will be understood that the number of appendages 304, and the number of markers 301 (provided that there are at least three) may vary without departing from the present teachings, where a greater number of markers 301 may increase the accuracy and/or precision of the determination of the pose of the magnetic field source attached to the constellation of markers 300, where an increase in the number of markers 301 results in an increase of the number of points in space from which the pose of the magnetic field source may be determined.

In some instances, the appendages 304 next to the mount 303 extend in a direction opposite from the appendages 304 that are furthest from the mount 303.

In some instances, the appendages 304 located on a first side of the frame 302 may be symmetrical with the appendages 304 located on a second opposite side of the frame 302.

For instance, as illustrated in FIG. 5, some of the frames 302 of the constellations of markers 300 have shorter appendages 304, the appendages 304 opposite to the end of the frame 302 where the mount 303 is located, than the others. The shorter appendages 304 (these shorter appendages opposite to the end of the frame 302 where the mount 303 is located) of some of the constellations of markers 300 of the set of the constellations of markers may fit into the space created by the longer appendages 304 (these longer appendages opposite to the end of the frame 302 where the mount 303 is located) of the other constellations of markers 300 of the set of constellations of markers 300. The result is that each constellation of markers 300 has a unique arrangement of markers (resulting in a unique signature) when compared to the other constellation of markers.

In some instances, the shape of each constellation of markers 300 may be registered in the system 100 (e.g. including the position of the markers 301 for each constellation of markers 300). This may facilitate the tracking of the markers 301 to determine the pose of the magnetic field sources.

### EXEMPLARY METHOD OF ADJUSTING POSES OF MAGNETIC FIELD SOURCES OF A MAGNETIC FIELD SOURCE SYSTEM:

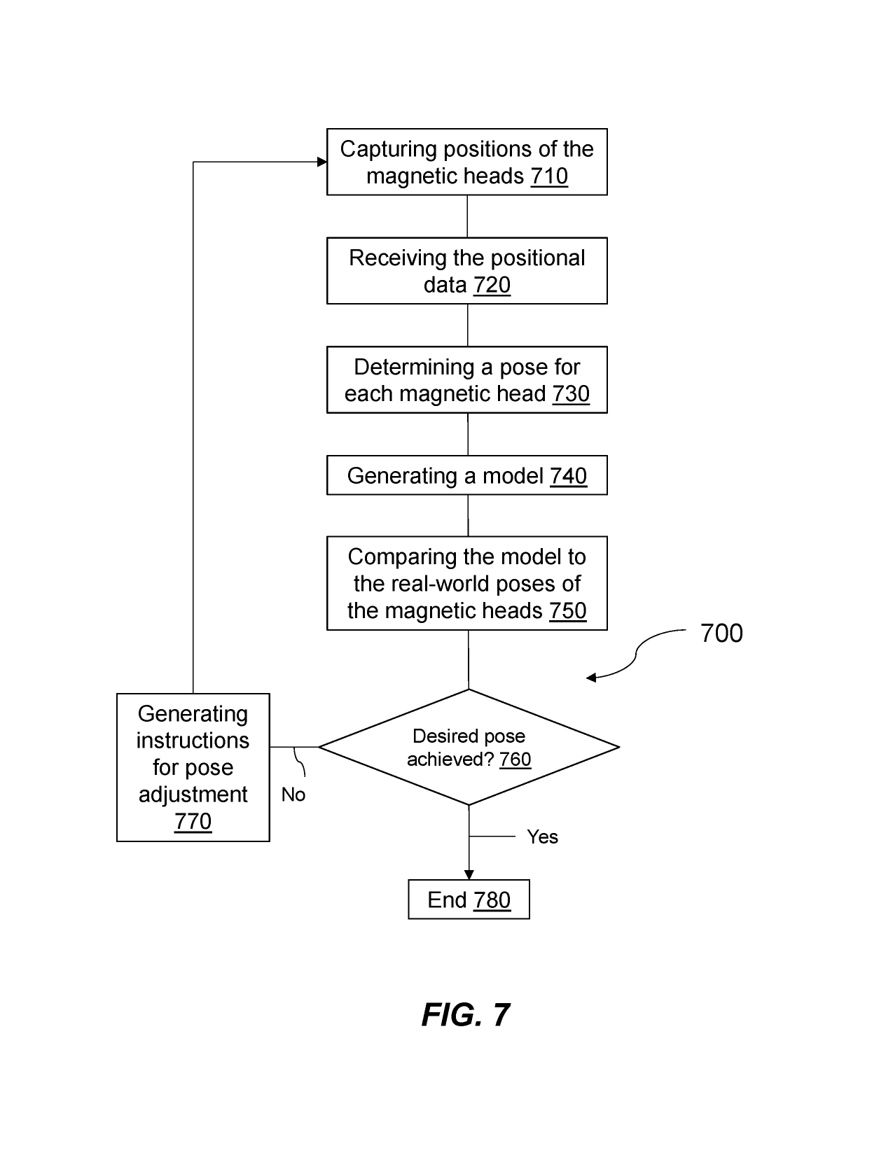

Reference is now made to FIG. 7, illustrating an exemplary method 700 of adjusting poses of magnetic field sources of a magnetic field source system. The method 700 may be performed by system 100 and position sensors 110, or any other system and/or position sensors in accordance with the present teachings.

Positional data of the magnetic field sources is generated by the one or more position sensors at step 710. The positional data includes information on, or to derive therefrom, a position and orientation (i.e. a pose).

For instance, when the position sensor(s) are camera(s), the camera(s) may capture in an image stream the magnetic field sources of the magnetic field source system. In these examples, the positional data is the image stream(s) generated by the camera(s). In some embodiments, when each of the constellations of markers is attached to a respective magnetic field source, the image stream may instead or also capture the constellations of markers.

In some instances, the positional data may include a position and orientation of an end effector of a robotic arm, where the position and orientation of the end effector may be corelated to the pose of the magnetic field source. The end effector may be contacting the magnetic field source.

The positional data is data generated by the one or more position sensors. The poses of the magnetic field sources may be derived from the positional data. As such, the positional data contains information indicative of the poses of the magnetic field sources.

The positional data, generated by the one or more position sensors, is received at step 720.

The poses of the magnetic field sources are determined at step 730 from the positional data related to the magnetic field sources.

In some instances, when constellations of markers are used and captured in the one or more image streams, the shapes of the different constellations of markers may be registered, such that performance of object recognition on an image stream accounts for the difference in dimensions of the different constellations of markers. As such, each marker object identified in an image stream can be related to its corresponding constellation of markers (e.g. by assigning an identifier in the metadata corresponding to the marker object that is related to the constellation of markers).

For instance, when the positional data is an image stream, the positional data may be analyzed to derive the pose of the magnetic field sources by performing object recognition of the objects appearing in the image stream. In one example, where constellations of markers are affixed to the magnetic field sources, an object recognition application program may be used to identify the markers appearing in the image stream. In some instances, the marker objects may be identified manually in the one or more image streams. The markers can be related to their respective magnetic field sources (e.g. by defining the objects appearing in image space corresponding to the markers, and defining in the metadata of the object data structure an identifier specific to that magnetic field source). In some instances, where the position of the markers on the frame of the constellation of markers is known, the positions of the marker objects appearing in the image space of the image stream may be mapped to defined configurations of constellations of markers, to associate the identified marker objects to their respective constellation of markers, and to the magnetic field source attached thereto.

Once the marker objects for a magnetic field source are identified in an image stream, their relationship with respect to one another in image space provides information on the position and orientation of the constellation of markers in three-dimensional space, and therefore the magnetic field source that is attached thereto. In some instances, where there are multiple cameras with different fields of view of the magnetic field sources, the relative positions of the markers objects in the different image streams, generated by the different cameras, can further be analyzed to improve the position of the markers, and their respective magnetic field source, in three-dimensional space, to determine the pose of the constellation of markers, and as a result, that of the magnetic field source.

The calculation of the pose of the magnetic field source from the positions of the marker objects in image space is repeated for each of the magnetic field sources. The calculated pose values can be stored in memory.

In some instances, a model of the three-dimensional representation of the magnetic field sources in the real world can be generated on a display to assist a user in visualizing the current pose of each of the magnetic field sources in the real world.

In some instances, where there is no constellation of markers affixed to the end of the magnetic field source, markers may instead be positioned on the outside surface of the magnetic field source (e.g. around a circumference of the magnetic field source). The markers on the outside surface of the magnetic field source may then be captured in one or more image streams. The marker objects may be identified in the image space of the image stream, and their relative position with respect to one another may be analyzed to determine the pose of the magnetic field source.

In some instances, when the positional data is generated by a position and orientation of an end effector of a robotic arm, the position and orientation of the robotic arm may be analyzed to determine therefrom the pose of the magnetic field source (e.g. where a difference between an at-rest position and orientation of the end effector can be mapped to the position and orientation of the magnetic field source).

A virtual three-dimensional model of the magnetic field sources, with a select pose defined for each of the magnetic field sources of the magnetic field source system, of the magnetic field source system is generated at step 740. The virtual model includes virtual representations of the magnetic field sources but with a select pose that is positioned and oriented with respect to the isocenter of the magnetic field source system. The virtual model of the magnetic field sources may be rendered on a display of the magnetic field source system (or of a client computer), in order for a user to visualize the select poses of the magnetic field sources.

In some instances, the virtual model may include, or be presented by, a set of values indicative of the target position and target orientation of each of the virtual magnetic field sources forming the virtual model.

In some instances, the position of the isocenter may be determined or repositioned as a function of the current real-world poses of the magnetic field sources of the magnetic field source system. In some instances, the select poses of the magnetic field sources of the virtual model of the magnetic field sources may also be adjusted as a function of the adjustment of the isocenter and the real-world poses of the magnetic field sources of the magnetic field source system.

In some embodiments, a virtual model of the magnetic field sources in their real-world poses may be rendered on the display, next to the select poses of the magnetic field sources appearing in the virtual model of the magnetic field sources, thereby illustrating the differences in positions and orientations between the real-world magnetic field sources and the respective magnetic field sources in their target positions and orientations.

For each of the magnetic field sources of the magnetic field source system, the real-world orientation value and real-world position value of the magnetic field source is compared to the target position value and the target orientation value for the magnetic field source at step 750.

When the orientation value of the real-world pose of a magnetic field source in x, y, and/or z is different from that of the select pose in x, y and/or z of the virtual counterpart of the magnetic field source, a difference in the x, y and z values for the magnetic field source is calculated to determine a difference in orientation between the real-world orientation of the magnetic field source and the target orientation for the same magnetic field source.

When the position value in x, y and/or z for the real-world pose is different from the position value in x, y and/or z for the select pose of the magnetic field source, a vector or difference in x, y and/or z is calculated between the real-world position of the magnetic field source and the target position for the same magnetic field source.

An evaluation of if the real-world magnetic field source has a pose that corresponds to the select pose is performed at step 760. The real-world pose has reached the select pose for that magnetic field source when the difference in orientation between the real-world magnetic field source and its virtual counterpart modeled in the select pose is 0 (or meets a value set by the system that is at a range from 0), and when the difference in position between the real-world magnetic field source and its virtual counterpart modeled in the select pose is 0.

The evaluation of if the real-world magnetic field source has a pose that corresponds to the select pose is repeated for each magnetic field source of the magnetic field source system.

If the real-world pose of each of the magnetic field sources of the magnetic field source system corresponds to their respective select pose, as defined in the three-dimensional virtual model of the magnetic field sources, then the poses of the real-world magnetic field sources are sufficiently calibrated and a further adjustment of the position and/or orientation of the magnetic field source(s) is not further required at step 780.

However, if the real-world pose of one or more of the magnetic field sources is different from its select pose as defined in the three dimensional virtual model of the magnetic field sources, then instructions are generated at step 770 to adjust the position and/or orientation of one or more of the magnetic field sources through a calculation of a rotation and/or translation to be carried out for one or more the magnetic field sources based on the difference in the real-world pose and the select pose for a magnetic field source, such that, following the application of the instructions, the pose of the real-world magnetic field source is closer to the select pose defined by the virtual model.

In some instances, the instructions may be or include commands for causing an actuator to adjust a position and/or orientation of one or more of the magnetic field sources in accordance with the instructions. When the actuator receives the commands, the actuator may cause a change in the orientation and/or position of the real-world magnetic field source to at least approach (in some cases, depending on the range of motion of the magnetic field source, correspond to) the select pose for the magnetic field source as set in the virtual model of the magnetic field sources. In some instances, one actuator may be provided for each of the magnetic field sources, where separate commands are sent to each of the actuators to cause a change of position and/or orientation of each magnetic field source having a real-world pose that diverges from the select pose as defined in the virtual model of the magnetic field sources.

In some instances, the instructions may cause a display of a visual indicator (on a display) for guiding an operator to manually adjust the orientation and/or position of one or more of the magnetic field sources. A visual indicator may be one or more arrows indicating the direction of the change of orientation and/or position required. A visual indicator may be an overlapping model of the magnetic field source with the select pose and the same magnetic field source with the real-world difference, where an operator may adjust the orientation and/or position of the magnetic field source until the two models (real-world pose and select pose) of the magnetic field source are aligned with respect to one another.

In some examples, the visual indicator may instead be a string of characters forming instructions to adjust the pose of the position and/or orientation of one or more of the magnetic field sources, corresponding to the difference between the real-world pose and select pose for each of the magnetic field sources.

In some instances, the instructions may be in the form of an audio indicator that is shared with the operator through a speaker (e.g. where the audio indicator may be verbal instructions guiding the operator in the adjustment of each of the magnetic field sources of the magnetic field source system) (e.g. “rotate clockwise until you hear a beep”).

It will be understood that other forms of instructions, in order to cause the real-world pose of one or more magnetic field sources to at least approach their respective select pose (either through a mechanism, or through manual actual of an operator) may be contemplated without departing from the present teachings.

Following an adjustment to the real-world pose of one or more of the magnetic field sources of the magnetic field source system, steps 710-760 may be repeated in order to determine if the adapted real-world poses of the magnetic field sources match the select poses of the magnetic field sources as defined in the virtual model of the magnetic field sources. Steps 710-770 may be further repeated cyclically until the real-world poses of the magnetic field sources match the select poses of the magnetic field sources as defined in the virtual model of the magnetic field sources.

In some instances, despite there being a difference between the real-world poses of the magnetic field sources and the select poses of the magnetic field sources as defined in the virtual model of the magnetic field sources, user input may be provided by an operator to stop the cyclic steps 710-770 (e.g. when the real-world poses of the magnetic field sources are sufficiently close to the select poses of the magnetic field sources as defined in the virtual model of the magnetic field sources).

In some embodiments, the magnetic field sources may have a limited range of motion that may prevent an adjustment of the real-world poses of the magnetic field sources such that the real-world poses of the magnetic field sources match the select poses of the magnetic field sources as defined in the virtual model of the magnetic field sources. In some instances, the limitations in the range of motion of the magnetic field sources may be entered into the system when generating the instruction(s) for adjustment of the real-world pose of one or more of the magnetic field sources at step 770.

In some embodiments, an adjustment to the orientation or configuration of the virtual model of the magnetic field sources may be performed in order to accommodate the limitations in the range of motion of one or more of the magnetic field sources. The adjustment to the virtual model may result in the virtual model being manipulated or modified to accommodate (e.g. oriented) the magnetic field source with a limited range of motion, in order for that magnetic field source to undergo limited changes to the real-world pose of that magnetic field source in order for that magnetic field source to arrive at the select pose set by the virtual model. The virtual model may be adjusted as a function of the magnetic field source(s) with greater range of motion, as well as those with a lesser range of motion. In some instances, the virtual model may be adjusted based on the ranges of motion of the magnetic field source to reach a configuration of the virtual model where the real-world magnetic field sources can be adjusted to arrive at a real-world pose configuration that is close or closest to the select poses of the magnetic field source set by the virtual model, while taking into consideration the limitations in the ranges of motion of the magnetic field sources of the magnetic field source system.

Method 700 may be performed when a magnetic field source system is installed in a new room (e.g. a room in a healthcare facility). The calibration of the pose of the magnetic field sources may be to offset slight irregularities in the room, such as an uneven floor, which may undermine the precision of the diagnostic, imagery and/or treatment procedures carried out by the magnetic field source system (where small errors in the expected pose of the magnetic field source(s), as defined in software running the magnetic field source system, and the real-world poses of the magnetic field sources, may reduce the efficacy of the medical intervention). The method 700 is therefore used to correct for differences in the poses of the magnetic field sources from their expected poses, as defined in the software controlling the magnetic field sources for carrying out a medical procedure, which can be the result of irregularities in the room or resulting from installation of the magnetic field source system.

In some implementations, method 700 may be carried out periodically to recalibrate the poses of the magnetic field sources over time. The recalibration may be performed to compensate for shifts in the structure of the building (e.g. the floor on which the magnetic field source system is installed) in which the magnetic field source system is installed, which can impact the poses of the magnetic field sources. The recalibration may also be performed to correct for changes in the poses of the magnetic field source that may result from regular use of the magnetic field source system or manipulation of the magnetic field source system.

Method 700 may be performed periodically to confirm that the magnetic field source system has maintained an adequate configuration for continued used.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is:

1. A method for guiding the positioning of magnetic field sources of a magnetic-field based system, the system comprising a plurality of magnetic field sources, comprising:
receiving positional data for each of the plurality of magnetic field sources;
determining a pose of each magnetic field source of the plurality of magnetic field sources from the positional and orientation data;
generating a computer model of a target layout of the plurality of magnetic field sources, defining a select pose for each of the plurality of magnetic field sources; and
for each magnetic field source of the plurality of magnetic field sources, comparing the select pose with the determined pose to calculate one or more of a rotation and translation of the magnetic field source; and
generating instructions to adjust one or more of a position and orientation of at least one of the plurality of the magnetic field sources from the calculated one or more of a rotation and translation, for causing the plurality of magnetic field sources to approach the target layout of the computer model.

2. The method as defined in claim 1, wherein the positional data for each of the plurality of magnetic field sources is generated from one or more cameras, wherein a combined field of view of the one or more cameras encompasses each of the plurality of magnetic field sources.

3. The method as defined in claim 2, wherein each magnetic field source of the plurality of magnetic field sources includes a constellation of markers attached thereto, and wherein the positional data is further generated from the constellation of markers captured by the one or more cameras.

4. The method as defined in claim 3, wherein supporting frames are joined to the plurality of magnetic field sources, each constellation of markers is joined to a supporting frame of the supporting frames configured to position at least three markers of the constellation of markers to provide information for defining the pose of each magnetic field source of the plurality of magnetic field sources, where each supporting frame of the supporting frames is joined to a different magnetic field source of the plurality of magnetic field sources.

5. The method as defined in claim 4, wherein one or more of the supporting frames has a different shape from other supporting frames of the one or more supporting frames.

6. The method as defined in claim 4, wherein the supporting frames are two dimensional.

7. The method as defined in claim 1, wherein the positional data for each of the plurality of magnetic field sources is generated from a robotic arm that is positioned to contact one or more of the plurality of magnetic field sources.

8. The method as defined in claim 1, wherein the plurality of magnetic field sources comprises three pairs of magnetic field sources.

9. The method as defined in claim 8, wherein the target layout defines a position of the plurality of magnetic field sources, wherein each pair of the three pairs is orthogonal with respect to each of the other two pairs of the three pairs.

10. The method as defined in claim 1, further comprising:
receiving further positional data for each of the plurality of magnetic field sources following an adjustment of the one or more of a position and orientation of the magnetic field source in accordance with the instructions;
determining an updated pose of each magnetic field source of the plurality of magnetic field sources from the positional data; and
for each magnetic field source of the plurality of magnetic field sources, comparing the select pose with the determined updated pose to calculate one or more of a further rotation and further translation of the magnetic field source; and
generating instructions to adjust one or more of the position and the orientation of at least one of the plurality of magnetic field sources from the corresponding calculated one or more of an additional rotation and additional translation, for causing the plurality of magnetic field sources to further approach the target layout.

11. The method as defined in claim 1, prior to the comparing, further comprising adjusting an orientation of the computer model of a target layout to align a computer magnetic field source of the model with a pose of the corresponding magnetic field source of the plurality of magnetic field sources.

12. A computing device for guiding the positioning of magnetic field sources of a magnetic-field based system, wherein the system comprises a plurality of magnetic field sources, the computing device comprising:
a processor; and
memory storing program code that, when executed by the processor, causes the processor to:
receive positional data for each of the plurality of magnetic field sources;
determine a pose of each magnetic field source of the plurality of magnetic field sources from the positional data;
generate a computer model of a target layout of the plurality of magnetic field sources, defining a select pose for each of the plurality of magnetic field sources; and
for each magnetic field source of the plurality of magnetic field sources, compare the select pose with the determined pose to calculate one or more of a rotation and translation of the magnetic field source; and
generate instructions to adjust one or more of a position and orientation of at least one of the plurality of the magnetic field sources from the calculated one or more of a rotation and translation, for causing the plurality of magnetic field sources to approach the target layout.

13. The computing device as defined in claim 12, wherein the positional data for each of the plurality of magnetic field sources is generated from one or more cameras, wherein a combined field of view of the one or more cameras encompasses each of the plurality of magnetic field sources.

14. The computing device as defined in claim 13, wherein each magnetic field source of the plurality of magnetic field sources includes a constellation of markers attached thereto, and wherein the positional data is further generated from the constellation of markers captured by the one or more cameras.

15. The computing device as defined in claim 14, wherein supporting frames are joined to the plurality of magnetic field sources, each constellation of markers is joined to a supporting frame of the supporting frames configured to position at least three markers of the constellation of markers to provide information for defining the pose of each magnetic field source of the plurality of magnetic field sources, where each supporting frame of the supporting frames is joined to a different magnetic field source of the plurality of magnetic field sources.

16. The computing device as defined in claim 15, wherein one or more of the supporting frames has a different shape from other supporting frames of the one or more supporting frames.

17. The computing device as defined in claim 15, wherein the supporting frames (substrates) are two dimensional.

18. The computing device as defined in claim 12, wherein the positional data for each of the plurality of magnetic field sources is generated from a robotic arm that is positioned to contact one or more of the plurality of magnetic field sources.

19. The computing device as defined in claim 12, wherein the plurality of magnetic field sources comprises three pairs of magnetic field sources.

20. A non-transitory computer-readable medium having stored thereon program instructions for guiding the positioning of magnetic field sources of a magnetic-field based system, the program instructions executed by a processing unit for:
receiving positional data for each of the plurality of magnetic field sources;
determining a pose of each magnetic field source of the plurality of magnetic field sources from the positional data;
generating a computer model of a target layout of the plurality of magnetic field sources, defining a select pose for each of the plurality of magnetic field sources; and
for each magnetic field source of the plurality of magnetic field sources, comparing the select pose with the determined pose to calculate one or more of a rotation and translation of the magnetic field source; and
generating instructions to adjust one or more of a position and orientation of at least one of the plurality of the magnetic field sources from the calculated one or more of a rotation and translation, for causing the plurality of magnetic field sources to approach the target layout.
